(19) United States
(12) Patent Application Publication
(10) Pub. No.: US 2017/0231901 A1
(43) Pub. Date: Aug. 17, 2017

(54) ADIPOSE DERIVED MESENCHYMAL STEM CELL COMPOSITIONS

(71) Applicants: Jiansheng MENG, Nantong (CN); Jiong WU, San Diego, CA (US); Feng LIN, San Diego, CA (US)

(72) Inventors: Jiansheng MENG, Nantong (CN); Jiong WU, San Diego, CA (US); Feng LIN, San Diego, CA (US)

(21) Appl. No.: 15/435,204

(22) Filed: Feb. 16, 2017

Related U.S. Application Data
(60) Provisional application No. 62/295,993, filed on Feb. 16, 2016.

(51) Int. Cl. A61K 8/98; A61Q 19/00; A61Q 19/08

(52) U.S. Cl. CPC A61K 8/981; A61Q 19/08; A61Q 19/00; A61K 2800/81; A61K 2800/91

(57) ABSTRACT

The invention provides compositions of matter, methods and treatment means for improving cosmetic appearance of skin and restoring aged or damaged skin to a healthy appearance. One embodiment, the invention teaches administration of autologous adipose derived mesenchymal stem cells that have been cultured under “activating conditions”. In one specific embodiment activation is performed prior to administration of cells into patient's skin. In another embodiment adipose derived cells are administered systemically, with localization of stem cells to skin by administration of a localizing agent, said localizing agent comprising either a peptide; a protein; or a photoceutical.

### CROSS REFERENCE TO RELATED APPLICATIONS

This application claims benefit of priority to U.S. Provisional Application No. 62/295,993 filed Feb. 16, 2016, the contents of which are hereby incorporated by reference.

### FIELD OF THE INVENTION

The invention belongs to field of cellular therapy, more specifically, the invention belongs to the utilization of adipose derived stem cells for stimulation of skin regeneration and antiaging.

### SUMMARY OF THE INVENTION

Various aspects of the invention of the invention relating to the above are enumerated in the following paragraphs:

Aspect 1. A method of treating skin so that the appearance of the skin is improved, said method comprised of: a) extracting adipose cells from said patient in need of skin improvement; b) obtaining the stromal vascular fraction of said adipose tissue; c) exposing said stromal vascular fraction of said adipose tissue to one or more conditions capable of enhancing regenerative activity of cells in said stromal vascular fraction of said adipose tissue; d) administering said cells back to the said patient.

Aspect 2. The method of Aspect 1, wherein said skin in need of improvement is selected from a group comprising of: a) aged skin; b) skin with wrinkles; c) sun damaged skin; d) scarred skin; e) hypopigmented skin; and f) skin damaged by skin disorders.

Aspect 3. The method of Aspect 1, wherein said adipose cells are extracted by liposuction.

Aspect 4. The method of Aspect 3, wherein liposuction and extraction of adipose cells, termed stromal vascular fraction cells is performed by the following steps: a) Using aseptic technique and with local anesthesia, the infraumbilical region is infiltrated with 0.5% Xylocaine with 1:200,000 epinephrine; b) After allowing 10 minutes for hemostasis, a 4mm cannula attached to a 60 cc Toomey syringe is used to aspirate 500 cc of adipose tissue in a circumincisional radiating technique; c) As each of 9 syringes are filled, said syringes are removed from the cannula, capped, and exchanged for a fresh syringe in a sterile manner within the sterile field; d) Using aseptic laboratory technique, the syringe-filled lipoaspirate are placed into two sterile 500 mL centrifuge containers and washed three times with sterile Dulbecco's phosphate-buffered saline to eliminate erythrocytes; e) ClyZyme/PBS (7mL/500 mL) is added to the washed lipoaspirate using a 1:1 volume ratio; f) The centrifuge containers are sealed and placed in a 37° C. shaking water bath for one hour then centrifuged for 5 min at 300 rcf; g) Following centrifugation, the stromal cells are resuspended within Isolyte in separate sterile 50 mL centrifuge tubes; g) The tubes are centrifuged for 5 min. at 300 rcf and the Isolyte is removed, leaving cell pellet; h) The pellets are resuspended in 40 ml of Isolyte, centrifuged again for 5 min at 300 rcf. The supernatant is again be removed; i) The cell pellets are combined and filtered through 100 μm cell strainers into a sterile 50 ml centrifuge tube and centrifuged for 5 min at 300 rcf and the supernatant removed, leaving the pelleted adipose stromal cells.

Aspect 5. The method of Aspect 1, wherein said adipose derived cells are cultured for expansion of mesenchymal stem cells.

Aspect 6. The method of Aspect 5, wherein said adipose derived cells are positively selected for a marker chosen from a group comprising of: a) CD105; b) CD73; c) CD44; d) CD90; e) VEGFR2; and f) TEM-1.

Aspect 7. The method of Aspect 5, wherein said adipose derived cells are negatively selected for markers chosen from a group comprising of: a) HLA-DR; b) CD45; and c) CD14.

Aspect 8. The method of Aspect 5, wherein said cells are grown in DMEM media supplement with antibiotics and fetal calf serum.

Aspect 9. The method of Aspect 8, wherein said fetal calf serum is added at a concentration of 10%.

Aspect 10. The method of Aspect 1, wherein said stimulation of regenerative factors is performed by culture for a period of 0.1-72 hours in the presence of an activator of toll like receptor (TLR)-3.

Aspect 11. The method of Aspect 10, wherein said TLR-3 receptor agonist is Poly IC.

Aspect 12. The method of Aspect 11, wherein said Poly IC is added to said culture at a concentration of 1-100 ng/ml.

Aspect 13. The method of Aspect 12, wherein said Poly IC is added to said culture at a concentration of approximately 10 ng/ml.

Aspect 14. The method of Aspect 10, wherein said Poly IC is added so said cells for a period of approximately 12 hours.

Aspect 15. The method of Aspect 1, wherein said enhancement of regenerative activity of said cells is endowed by exposure to laser irradiation.

Aspect 16. The method of Aspect 15, wherein said laser irradiation is provided in at least one wavelength, said wavelength in a range between about 620 nanometers and about 1070 nanometers.

Aspect 17. The method of Aspect 16, wherein said laser irradiation is provided for a sufficient time and energy intensity to augment activity of said cells containing a concentrated stem cell population.

Aspect 18. The method of Aspect 17, wherein said activity of said administered cells is selected from a group comprising of: a) enhanced cytokine production; b) enhanced ability to differentiate into cells of the pulmonary architecture; c) augmented ability to produce antiapoptic factors; d) increased angiogenic activity; e) inhibition of inflammatory cytokine production; and f) inhibition of fibrotic activity.

Aspect 19. The method of Aspect 17 wherein said laser irradiation is administered by a light source between approximately 100 .mu.W/cm.sup.2 to approximately 10 W/cm.sup.2.

Aspect 20. The method of Aspect 1, wherein said cells are administered intradermally.

Aspect 21. The method of Aspect 1, wherein said cells are administered systemically.

Aspect 22. The method of Aspect 21, wherein said patient receiving cells administered systemically is further treated with an agent or plurality of agents capable of achieving stem cell retention to the dermal area where therapeutic effects are desired.

Aspect 23. The method of Aspect 22, wherein said agents capable of achieving stem cell retention are selected from a group comprising of: a) stromal derived factor-1 (SDF-1); b) vascular endothelial growth factor (VEGF); c) epidermal growth factor (EGF); d) platelet rich plasma; e) brain derived neurotrophic factor (BDNF), f) platelet derived growth factor (PDGF); and g) low level laser irradiation.

Aspect 24. A method of treating skin so that the appearance of the skin is improved, said method comprised of: a) obtaining cells with regenerative properties; b) exposing said cells to one or more conditions capable of enhancing regenerative activity of said cells cells; d) administering said cells to said patient in need of treatment.

Aspect 25. The method of Aspect 24, wherein said cells with regenerative properties are autologous, allogeneic, or xenogeneic to the recipient.

Aspect 26. The method of Aspect 24, wherein said cells with regenerative properties are obtained from tissues selected from a group of tissues comprising of: a) adipose tissue; b) bone marrow; c) muscle; d) mobilized peripheral blood; e) hair follicle; f) teeth; and g) periventricular fluid.

Aspect 27. The method of Aspect 26, wherein cells possessing regenerative potential are mesenchymal.

Aspect 28. The method of Aspect 27, wherein said cells possess ability to produce cytokines selected from a group comprising of: a) FGF-alpha; b) FGF-beta; c) FGF-V; d) EGF; e) IGF; f) VEGF; g) SDF-1; h) PDGF-1; and i) BDNF.

Aspect 29. The method of Aspect 26 wherein the step of processing said cells from said adipose tissue so as to concentrate said stem cell component is performed through treatment with an enzyme capable of enriching for stromal vascular fraction cells.

Aspect 30. The method of Aspect 26, wherein the step of processing said cells from said bone marrow tissue to concentrate said stem cell component is performed through removal of erythrocytes and granulocytes by use of a density gradient.

Aspect 31. The method of Aspect 26, wherein the step of processing said cells from said muscle tissue so as to concentrate said stem cell component is performed through treatment with an enzyme capable of enriching for cells expressing a marker selected from a group of markers comprising of CD13, CD34, CD56 and CD117.

Aspect 32. The method of Aspect 26, wherein the step of processing said cells from said mobilized blood so as to concentrate said stem cell component is performed through leukopheresis of a patient who has been mobilized by a mobilizing agent selected from a group comprising of: G-CSF, Mozobil, VEGF, or parathyroid hormone.

Aspect 33. The method of Aspect 32, wherein said cells purified by leukopheresis are further selected for expression of CD34.

Aspect 34. The method of Aspect 26, wherein the step of processing said cells from said hair follicle tissue so as to concentrate said stem cell component is performed through an ex vivo expansion step, selecting for cells expressing CD117.

Aspect 35. The method of Aspect 26, wherein the step of processing said cells from said tooth tissue so as to concentrate said stem cell component is performed through an ex vivo expansion step, selecting for cells expressing CD117.

Aspect 36. The method of Aspect 26, wherein enrichment of stem cell component of said tissue is performed through selection of cells expressing the enzyme aldehyde dehydrogenase.

Aspect 37. The method of Aspect 26, wherein enrichment of stem cell component of said tissue is performed through selection of cells expressing the side population phenotype.

Aspect 38. The method of Aspect 37, wherein said cells are purified by ability to efflux Rhodamine 123.

Aspect 39. The method of Aspect 26, wherein said periventricular stem cells are expanded and selected for cells expressing an antigen selected from the group comprising of: CD117, c-kit, and Oct-4.

Aspect 40. The method of Aspect 24, wherein said condition capable of stimulating regenerative activity is exposure to low level laser irradiation.

Aspect 41. The method of Aspect 40, wherein said laser irradiation is provided in at least one wavelength, said wavelength in a range between about 620 nanometers and about 1070 nanometers.

Aspect 42. The method of Aspect 40, wherein said laser irradiation is provided for a sufficient time and energy intensity to augment activity of said cells containing a concentrated stem cell population.

Aspect 43. The method of Aspect 24, wherein said activity of said administered cells is selected from a group comprising of: a) enhanced cytokine production; b) enhanced ability to differentiate into cells of the dermal architecture; c) augmented ability to produce antiapoptic factors to cells of the dermal architecture; d) increased angiogenic activity; e) inhibition of inflammatory cytokine production; and f) inhibition of fibrosis.

Aspect 44. The method of Aspect 40 wherein said laser irradiation is administered by a light source between approximately 100 .mu.W/cm.sup.2 to approximately 10 W/cm.sup.2.

Aspect 45. The method of Aspect 24, wherein said stimulation of regenerative factors is performed by culture for a period of 0.1-72 hours in the presence of an activator of toll like receptor (TLR)-3.

Aspect 46. The method of Aspect 45, wherein said TLR-3 receptor agonist is Poly IC.

Aspect 47. The method of Aspect 46, wherein said Poly IC is added to said culture at a concentration of 1-100 ng/ml.

Aspect 48. The method of Aspect 47, wherein said Poly IC is added to said culture at a concentration of approximately 10 ng/ml.

Aspect 49. The method of Aspect 46, wherein said Poly IC is added so said cells for a period of approximately 12 hours.

Aspect 50. A Aspect of treating skin so that the appearance of the skin is improved, said method comprised of: a) extracting adipose cells from said patient in need of skin improvement; b) obtaining the stromal vascular fraction of said adipose tissue; c) exposing said stromal vascular fraction of said adipose tissue to one or more conditions capable of enhancing regenerative activity of cells in said stromal vascular fraction of said adipose tissue; d) administering said cells back to the said patient.

Aspect 51. The method of Aspect 50 wherein said stromal vascular fraction cells are enriched for monocytic content.

Aspect 52. The method of Aspect 51, wherein said purification for monocytic content is achieved by positive selection for a monocytic marker.

Aspect 53. The method of Aspect 52, wherein said positive selection marker is selected from a group comprising of: a) CD14; b) CD73; c) CD68; and d) CD163.

Aspect 54. A method of treating skin so that the appearance of the skin is improved, said method comprised of: a) extracting adipose cells from said patient in need of skin improvement; b) obtaining the stromal vascular fraction of said adipose tissue; c) exposing said stromal vascular fraction of said adipose tissue to apoptotic bodies derived at a sufficient time and concentration so as to augment skin rejuvenating properties of said adipose stromal vascular fraction; and d) administering said adipose stromal vascular fraction cells back to the said patient.

Aspect 55. The method of Aspect 54, wherein said apoptotic bodies are derived from a cellular source.

Aspect 56. The method of Aspect 54, wherein said apoptotic bodies are artificial structures resembling apoptotic bodies.

Aspect 57. The method of Aspect 55, wherein said cellular source is peripheral blood mononuclear cells.

Aspect 58. The method of Aspect 57, wherein said peripheral blood mononuclear cells are treated with heat at a sufficient time and temperature to induce apoptosis.

Aspect 59. The method of Aspect 58, wherein said peripheral blood mononuclear cells are treated for a minimum of 10 minutes and for a maximum of 24 hours at a temperature of 40 to 42 degree Celsius.

Aspect 60. The method of Aspect 59, wherein said peripheral blood mononuclear cells are treated for a minimum of 30 minutes and for a maximum of 2 hours at a temperature of 40 to 42 degree Celsius.

Aspect 61. The method of Aspect 60, wherein said peripheral blood mononuclear cells are treated for 1 hour at a temperature of 41 degree Celsius.

Aspect 62. The method of Aspect 57, wherein said peripheral blood mononuclear cells are treated with ultraviolet irradiation at a sufficient time and temperature to induce apoptosis.

Aspect 63. The method of Aspect 62, wherein said UV irradiation is administered to said cells at a dose ranging from 1,200 joules/m² to 20,000 joules/m².

Aspect 64. The method of Aspect 63, wherein said UV irradiation is administered to said cells at a dose ranging from 5000 joules/m² to 15,000 joules/m².

Aspect 65. The method of Aspect 62, wherein said UV irradiation is administered to said cells at a dose ranging from 1,200 joules/m² to 20,000 joules/m².

### BRIEF DESCRIPTION OF THE DRAWINGS

FIG. 1 is a bar graph showing an increase in skin regenerative factor KGF was observed.

FIG. 2 is a bar graph showing an increase in skin regenerative factor EGF was observed.

FIG. 3 is a bar graph showing an increase in skin regenerative factor FGF-beta was observed.

### DETAILED DESCRIPTION

The invention teaches means of augmenting regenerative activity of adipose derived cells for enhancing their cosmetic properties. In one embodiment the invention provides for the use of innate immune stimulatory conditions in the pretreatment of stromal vascular fraction cells prior to administration for cosmetic use in order to augment regenerative properties. In another embodiment of the invention, stromal vascular fraction cells, or mesenchymal stem cells derived thereof, are administered intravenously, followed by laser or other chemoattractant therapy to areas of the skin where selective regeneration is desired.

In order to allow the comprehension and practice of this invention, the meanings of some terms and expressions as they are used in the context of the invention are included.

“Skin” is understood to be the layers which comprise it, from the uppermost layer or stratum corneum to the lowermost layer or hypodermis, both inclusive. These layers are composed of different types of cells such as keratinocytes, fibroblasts, melanocytes, mast cells, neurons and/or adipocytes among others. The term “skin” also comprises the scalp.

“Treatment”, according to its use in the context of this specification when it is not accompanied by the qualifications “cosmetic, non-therapeutic”, means the administration of a compound according to the invention to alleviate or eliminate a disease or disorder or reduce or eliminate one or more symptoms associated with said disease or disorder. The term “treatment” also covers alleviating or eliminating physiological consequences of the disease or disorder. When the term “treatment” is accompanied by the qualifications “cosmetic, non-therapeutic”, it refers to the application of the compound to the skin, hair and/or mucous membranes in particular with the aim of improving the cosmetic qualities of the skin, hair and/or mucous membranes such as, for example and not restricted to, their level of hydration, elasticity, firmness, shine, tone or texture, among others. The term “care” in this invention refers to the maintenance of the qualities of the skin, hair and/or mucous membranes. Said qualities are subject to being improved or maintained by cosmetic treatment and/or care of the skin, hair and/or mucous membranes both in healthy subjects as well as in those which present diseases and/or disorders of the skin and/or mucous membranes such as, for example and not restricted to, ulcers and injuries to skin, psoriasis, dermatitis, acne or rosacea, among others.

“Prevention”, as used in this invention, refers to the ability of a compound of the invention to prevent, delay or hinder the appearance or development of a disease or disorder before its appearance or improve the cosmetic qualities of the skin, mucous membranes and/or hair.

“Aging” refers to the changes experienced by the skin with age (chronoaging) or through exposure to the sun (photoaging) or to environmental agents such as tobacco smoke, extreme climatic conditions of cold or wind, chemical contaminants or pollutants, and includes all the external visible and/or perceptible changes through touch, such as and not restricted to, the development of discontinuities on the skin such as wrinkles, fine lines, expression lines, stretch marks, furrows, irregularities or roughness, increase in the size of pores, loss of hydration, loss of elasticity, loss of firmness, loss of smoothness, loss of the capacity to recover from deformation, loss of resilience, sagging of the skin such as sagging cheeks, the appearance of bags under the eyes or the appearance of a double chin, among others, changes to the color of the skin such as marks, reddening, bags or the appearance of hyperpigmented areas such as age spots or freckles among others, anomalous differentiation, hyperkeratinization, elastosis, keratosis, hair loss, orange-peel skin, loss of collagen structure and other histological changes of the stratum corneum, of the dermis, epidermis, vascular system (for example the appearance of spider veins or telangiectasias) or of those tissues close to the skin, among others. The term “photoaging” groups together the set of processes due to the prolonged exposure of the skin to ultraviolet radiation which result in the premature aging of the skin, and it presents the same physical characteristics as aging, such as and not restricted to, flaccidity, sagging, changes to the color or irregularities in the pigmentation, abnormal and/or excessive keratinization. The sum of various environmental factors such as exposure to tobacco smoke, exposure to pollution, and climatic conditions such as cold and/or wind also contribute to the aging of the skin.

“Senescence” is understood to be the changes to the organism as it ages after maturity and which affect both the cells and their functions and the whole organism. “Cell senescence” is understood to be the loss of the cells for their replication capacity by themselves, resulting in a degradation of the cells over time. Cell senescence is particularly important in cells with the capacity to replicate in the central nervous system, such as astrocytes, endothelial cells and fibroblasts which play a key role in age-related diseases such as Alzheimer's disease, Parkinson's disease, Huntington's disease, and stroke; cells with finite replicative capacity in the integument, including fibroblasts, sebaceous gland cells, melanocytes, keratinocytes, Langerhans cells, and hair follicle cells which play a key role in age-related diseases in the integument, such as dermal atrophy, elastolysis, wrinkles, sebaceous gland hyperplasia, senile lentigo, graying and hair loss, chronic skin ulcers, and age-related deterioration of the wound healing capacity; cells with finite replicative capacity in joint cartilage, such as chondroctyes and synovial fibroblasts which play a key role in degenerative joint diseases; cells with finite replicative capacity in the bone, such as osteoblasts, bone marrow stromal fibroblasts and osteoprogenitor cells which play a key role in osteoporosis; cells with finite replicative capacity in the immune system such as B and T lymphocytes, monocytes, neutrophils, eosinophils, basophils, NK cells and their respective progenitors, which can play a key role in the age-related deterioration of the immune system; cells with finite replicative capacity in the vascular system, including endothelial cells, smooth muscle cells, and adventitial fibroblasts which can play a key role in age-related diseases of the vascular system including atherosclerosis, calcification, thrombosis, and aneurisms; and cells with finite replicative capacity in the eye, such as the pigmented epithelium and vascular endothelial cells which can play an important role in age-related macular degeneration.

The inventors unexpectedly found that exposure of adipose derived stromal vascular fraction cells to various stimulators of innate immune responses leads to upregulation in regenerative activity as demonstrated by increased proliferation, cytokine production, as well as stimulation of fibroblast growth. Said innate stimulators of immunity include members of the toll like receptor (TLR) family. TLRs are known to be associated with recognition of tissue injury signals, said signals primarily found in intracellular stores and released upon tissue injury. In contrast to programmed cell death, or apoptosis, which is a physiological type of cell death, necrosis is considered a pathological type of cell death. Specifically, certain times and anatomical locations of the body are associated with natural cell death, examples of this include loss of neurons, or “pruning” during development, or deletion of T cell precursors, termed thymocytes, while positive and negative selection are occurring in the thymus. In both of these situations, cells die by apoptosis. The process of apoptosis is associated with activation of caspases, which cleave the DNA, as well as packaging of intracellular compartments into membrane vesicles. Since the intracellular compartments are not exposed to plasma, the “danger” signals are never exposed to the immune system. Additionally, during apoptosis, the cellular membrane flips, in that the intracellular facing side of the membrane, which is rich in phosphatidylserine, flips to the outside, whereas the extracellular facing side, which is rich in phosphatidylcholine flips to face the intracellular compartment. It is well known that phosphatidylserine, when on the outside of the cell, binds to receptors which inhibit inflammation and immune function [1-3]. The importance of phosphatidylserine in inhibiting inflammatory responses can be seen in studies in which artificially generated phosphatidylserine containing liposomes have been shown to inhibit inflammatory responses. For example, Shi et al demonstrated that subsequent to treatment with phosphatidylserine bearing liposomes, murine DCs display reduced expression of MHC II, CD80, CD86 and CD40, but increased programmed death ligand-1 (PD-L1 and PD-L2); and increased IL-10 and inhibited IL-12 cytokine production. Phosphatidylserine -treated DCs exhibit normal endocytic function, but ability to stimulate allogeneic T cells is reduced, similar to immature dendritic cell. Treatment of DCs with phosphatidylserine liposomes also suppressed DNCB induced CD4 +T cell proliferation and IFN-gamma production. Addition of exogenous IL-12p70 during the DC-T cell co-culture restored their IFN-gamma production. Furthermore, phosphatidylserine -treated DCs enhance the ratio of CD4(+) CD25(high)Foxp3(+) T cells to CD4(+) T cells and PD-1 expression on CD4(+) T cells [4].

In one embodiment of the invention, adipose derived mesenchymal stem cells are treated with a TLR agonist at a sufficient concentration and duration to augment production of skin regenerative proteins, and subsequently administered to the patient intradermally. One specific TLR agonist that was found useful for the practice of the invention is zymosan. This is a TLR2-binding glucan with repeating glucose units connected by (3-1,3-glycosidic linkages that is prepared from yeast cell wall and consists of protein-carbohydrate complexes. In one specific embodiment, mesenchymal stem cells are derived from stromal vascular fraction cells. Said stromal vascular fraction cells may be obtained by a variety of means, including, in one embodiment, the following: a) Using aseptic technique and with local anesthesia, the infraumbilical region is infiltrated with 0.5% Xylocaine with 1:200,000 epinephrine; b) After allowing 10 minutes for hemostasis, a 4 mm cannula attached to a 60 cc Toomey syringe is used to aspirate 500 cc of adipose tissue in a circumincisional radiating technique; c) As each of 9 syringes are filled, said syringes are removed from the cannula, capped, and exchanged for a fresh syringe in a sterile manner within the sterile field; d) Using aseptic laboratory technique, the syringe-filled lipoaspirate are placed into two sterile 500 mL centrifuge containers and washed three times with sterile Dulbecco's phosphate-buffered saline to eliminate erythrocytes; e) ClyZyme/PBS (7 mL/500 mL) is added to the washed lipoaspirate using a 1:1 volume ratio; f) The centrifuge containers are sealed and placed in a 37° C. shaking water bath for one hour then centrifuged for 5 min at 300 rcf; g) Following centrifugation, the stromal cells are resuspended within Isolyte in separate sterile 50 mL centrifuge tubes; g) The tubes are centrifuged for 5 min. at 300 rcf and the Isolyte is removed, leaving cell pellet; h) The pellets are resuspended in 40 ml of Isolyte, centrifuged again for 5 min at 300 rcf. The supernatant is again be removed; i) The cell pellets are combined and filtered through 100 μm cell strainers into a sterile 50 ml centrifuge tube and centrifuged for 5 min at 300 rcf and the supernatant removed, leaving the pelleted adipose stromal cells. Other techniques for isolation of stromal vascular fraction are well known in the art, for example, those used by the following references [5-9]. Additionally, stromal vascular fraction may be isolated using point of care devices such as the following: (1) PNC's Multi Station, (2) CHA Biotech Cha-Station, (3) Cytori Celution 800/CRS System, and (4) Medi-Khan's Lipokit with MaxStem [10]. Said stromal vascular fraction cells may subsequently be grown in liquid culture to selectively purify mesenchymal stem cell component. Said mesenchymal stem cell cultures are allowed to incubate for a period of approximately 72 hours or until reaching confluence. Media useful for the practice of the invention include DMEM, RPMI, AIM-V, OPTI-MEM, and EMEM. Media may be supplemented with nutrients or other factors to maintain viability of cells. Subsequent to culture, said media is harvested and utilized as a component of production of cosmetic. Activation of said stromal vascular fraction derived mesenchymal stem cells may be performed by incubation with 5 or 10 micrograms per ml of zymosan. Therapeutic activities may be assessed by production of growth factors associated with skin regeneration such as keratinocyte growth factor (KGF). Other growth factors of interest include: a) Interleukin-1 beta; b) Interleukin-6; c) alpha-2-Macroglobulin; d) Midkine; e) Chemokine (C-X-C motif) ligand 1 Chemokine (C-X-C motif) ligands; f) Chemokine (C-X-C motif) ligand 2; g) Chemokine (C-X-C motif) ligand 5; h) Chemokine (C-X-C motif) ligand 6; i) Interleukin-8; j) Chemokine (C-X-C motif) ligand 16; k) Chemokine (C-C motif) ligand 2; 1)Chemokine (C-C motif) ligand 8; m) WNT1-inducible-signaling pathway protein 2; n) Fibroblast growth factor 9; o) Platelet-derived growth factor D; p) Vascular endothelial growth factor A; and q) Growth differentiation factor 15.

In one embodiment, Lasers (Light amplification by stimulated emission of radiation) [11] are used to stimulate growth factor secretion properties to stromal vascular fraction cells, or purified mesenchymal stem cells prior to injection for facial regeneration. For the practice of the invention, a background is provided on Lasers [12-14]. Many applications of lasers are considered “high energy” because of their intensity, which ranges from about 10-100 Watts. For the practice of the current invention low level lasers (LLL) that elicits effects through non-thermal means is embodied. Use of LLL has previously been reported in regenerative medicine with the work of Mester et al who in 1967 reported non-thermal effects of lasers on mouse hair growth [15], however, this patent application is the first report of its use through the selective augmentation of stromal vascular fraction, and purified mesenchymal stem cell potency. For the practice of the invention, means of accelerating wound healing from previous publications [16], may be utilized by applying similar energy field and parameters to stromal vascular fraction cells, or expanded mesenchymal cells thereof in vitro prior to in vivo administration. Conversely, administration of LLL may be performed to skin subsequent to introduction of said stromal vascular fraction or purified mesenchymal stem cells thereof. Numerous in vitro and in vivo studies may be utilized to guide one of skill in the art in combining LLL with stromal vascular fraction cells or mesenchymal stem cells derived thereof.

For the practice of the invention, the definition of LLL therapy will be a modification of the proposed definition of Posten et al [17]: a) Power output of laser being 10(-3) to 10(-1) Watts; b) Wavelength in the range of 300-10,600 nm; c) Pulse rate from 0, meaning continuous to 5000 Hertz; d) intensity of 10(2)-10(0) W/cm(2) and dose of 10(-2) to 10(2) J/cm(2) . Most common methods of administering LLL radiation include lasers such as ruby (694 nm), Ar (488 and 514 nm), HeNe (632.8 nm), Krypton (521, 530, 568, and 647 nm), Ga—Al—As (805 or 650 nm), and Ga—As (904 nm). Perhaps one of the most distinguishing features of LLL therapy as compared to other photoceutical modalities is that effects are mediated not through induction of thermal effects but rather through a process that is still not clearly defined called “photobiostimulation”. It appears that this effect of LLL is not depend on coherence, and therefore allows for use of non-laser light generating devices such as inexpensive Light Emitting Diode (LED) technology [18]. Without being bound by theory, the combination of LLL with stem cells derived from adipose tissue aims to address the following biological manipulations: augmentation of cellular ATP levels [19], manipulation of inducible nitric oxide synthase (iNOS) activity [20, 21], suppression of inflammatory cytokines such as TNF-alpha, IL-1beta, IL-6 and IL-8 [22-26], upregulation of growth factor production such as PDGF, IGF-1, NGF and FGF-2 [26-29], alteration of mitochondrial membrane potential [19, 30-32] due to chromophores found in the mitochondrial respiratory chain [33, 34] as reviewed in [35], stimulation of protein kinase C (PKC) activation [36], manipulation of NF-kB activation [37], direct bacteriotoxic effect mediated by induction of reactive oxygen species (ROS) [38], modification of extracellular matrix components [39], stimulation of mast cell degranulation [40], and upregulation of heat shock proteins [41]. Studies of interest to guide one of skill in the art include: use of a helium neon (HeNe) laser to generate a visible red light at 632.8 nm for treatment of porcine granulosa cells. The upregulation of metabolic and hormone-producing activity of the cells when exposed for 60 seconds to pulsating low power (2.8 mW) irradiation was achieved [42]. The possibility of modulating biologically-relevant signaling proteins by LLL was further demonstrated in a study using an energy dose of 1.5 joules/cm(2) in cultured keratinocytes. Administration of HeNe laser emitted light resulted in upregulated gene expression of IL-1 and IL-8 [43]. Production of various growth factors in vitro suggests the possibility of enhanced cellular mitogenesis and mobility as a result of LLL treatment. Using a diode-based method to generate a similar wavelength to the HeNe laser (363 nm), Mvula et al reported in two papers that irradiation at 5 J/cm(2) of adipose derived mesenchymal stem cells resulted in enhanced proliferation, viability and expression of the adhesion molecule beta-1 integrin as compared to control [44, 45]. In agreement with possible regenerative activity based on activation of stem cells, other studies have used an in vitro injury model to examine possible therapeutic effects. Migration of fibroblasts was demonstrated to be enhanced in a “wound assay” in which cell monolayers are scraped with a pipette tip and amount of time needed to restore the monolayer is used as an indicator of “healing”. The cells exposed to 5 J/cm(2) generated by an HeNe laser migrated rapidly across the wound margin indicating a stimulatory or positive influence of phototherapy. Higher doses (10 and 16 J/cm(2)) caused a decrease in cell viability and proliferation with a significant amount of damage to the cell membrane and DNA [46]. In order to examine whether LLL may positively affect healing under non-optimal conditions that mimic clinical situations treatment of fibroblasts from diabetic animals was performed. It was demonstrated that with the HeNe laser dosage of 5 J/cm(2) fibroblasts exhibited an enhanced migration activity, however at 16 J/cm(2) activity was negated and cellular damage observed [47]. Thus from these studies it appears that energy doses from 1.5 joules/cm2 to 5 joules/cm2 are capable of eliciting “biostimulatory effects” in vitro in the HeNe-based laser for adherent cells that may be useful in regeneration such as fibroblasts and mesenchymal stem cells.

Studies have also been performed in vitro on immunological cells the frequency, duration, and wavelength of these laser interventions is applicable to adipose stromal vascular fraction cells, as well as mesenchymal stem cells derived thereof. High intensity HeNe irradiation at 28 and 112 J/cm(2) of human peripheral blood mononuclear cells, a heterogeneous population of T cells, B cells, NK cells, and monocytes has been described to induce chromatin relaxation and to augment proliferative response to the T cell mitogen phytohemagluttin [48]. In PBMC, another group reported in two papers that interleukin-1 alpha (IL-1 alpha), tumor necrosis factor-alpha (TNF-alpha), interleukin-2 (IL-2), and interferon-gamma (IFN-gamma) at a protein and gene level in human peripheral blood mononuclear cells (PBMC) was increased after HeNe irradiation at 18.9 J/cm(2) and decreased with 37.8 J/cm(2) [49, 50]. Stimulation of human PBMC proliferation and murine splenic lymphocytes was also reported with HeNe LLL [51, 52]. In terms of innate immune cells, enhanced phagocytic activity of murine macrophages have been reported with energy densities ranging from 100 to 600 J/cm(2), with an optimal dose of 200 J/cm(2) [53]. Furthermore, LLL has been demonstrated to augment human monocyte killing mycobacterial cells at similar densities, providing a functional correlation [54]. In one embodiment of the invention, PBMC treated by LLL or stimulated by a TLR agonist are cocultured with stromal vascular fraction derived mesenchymal stem cells. Subsequent to a period of culture, said stromal vascular fraction derived mesenchymal stem cells are removed and purified, and subsequently administered intradermally for therapeutic effect on skin regeneration.

In some embodiments, the use of LLL is applied with the aim of achieving a systemic effect, while administration of stromal vascular fraction cells, or mesenchymal stem cells derived thereof is performed locally. Examples of conditions and parameters of LLL practice with the aim of achieving systemic effects includes studies previously performed for conditions such as sinusitis [55], arthritis [56, 57], or wound healing [58], which are incorporated by reference. In some situations in vitro induction of inflammatory response is cells is desired, with the intent that subsequent in vivo administration will induce a rebound anti-inflammatory effect. The specific frequencies and duration of exposure to induce proinflammatory agents such as TNF-alpha or IL-1 is provided in the following, which are incorporated by reference [49, 50]. In some embodiments, LLL is substituted for, for the practice of the invention, with ozonation of blood [59-61]. In vitro studies have demonstrated that ozone is a potent oxidant and inducer of cell apoptosis and inflammatory signaling [62-64]. In one embodiment the conditions of the previously cited references are used to generated apoptotic bodies of stromal vascular fractions, or mesenchymal stem cells derived thereof. Specifically, the invention provides that said apoptotic bodies will induce more potent anti-oxidant enzyme activity such as elevations in Mg-SOD and glutathione-peroxidase levels, as well as diminishment of inflammation-associated pathology as compared to previous systems using whole blood ozonation [65-68].

Systems and frequencies are referenced from the literature to provide conditions useful for the practice of the invention in which stromal vascular fraction and mesenchymal stem cells derived thereof may be utilized. Surinchak et al reported in a rat skin incision healing model that wounds exposed HeNe radiation of fluency 2.2 J/cm(2) for 3 min twice daily for 14 days demonstrated a 55% increase in breaking strength over control rats. Interestingly, higher doses yielded poorer healing [69]. This application of laser light was performed directly on shaved skin. In a contradictory experiment, it was reported that rats irradiated for 12 days with four levels of laser light (0.0, 0.47, 0.93, and 1.73 J/cm(2)) a possible strengthening of wounds tension was observed at the highest levels of irradiation (1.73 J/cm(2)), however it did not reach significance when analyzed by resampling statistics [70]. In another wound-healing study Ghamsari et al reported accelerated healing in the cranial surface of teats in dairy cows by administration of HeNe irradiation at 3.64 J/cm2 dose of low-level laser, using a helium-neon system with an output of 8.5 mW, continuous wave [71]. Collagen fibers in LLLT groups were denser, thicker, better arranged and more continuous with existing collagen fibers than those in non-LLLT groups. The mean tensile strength was significantly greater in LLLT groups than in non-LLLT groups [72]. In the random skin flap model, the use of He-Ne laser irradiation with 3 J/cm(2) energy density immediately after the surgery and for the four subsequent days was evaluated in 4 experimental groups: Group 1 (control) sham irradiation with He-Ne laser; Group 2 irradiation by punctual contact technique on the skin flap surface; Group 3 laser irradiation surrounding the skin flap; and Group 4 laser irradiation both on the skin flap surface and around it. The percentage of necrotic area of the four groups was determined on day 7-post injury. The control group had an average necrotic area of 48.86%; the group irradiated on the skin flap surface alone had 38.67%; the group irradiated around the skin flap had 35.34%; and the group irradiated one the skin flap surface and around it had 22.61%. All experimental groups reached statistically significant values when compared to control [73]. Quite striking results were obtained in an alloxan-induced diabetes wound healing model in which a circular 4 cm(2) excisional wound was created on the dorsum of the diabetic rats. Treatment with HeNe irradiation at 4.8 J/cm(2) was performed 5 days a week until the wound healed completely and compared to sham irradiated animals. The laser-treated group healed on average by the 18th day whereas, the control group healed on average by the 59th day [74]. In addition to mechanically-induced wounds, beneficial effects of LLL have been obtained in burn-wounds in which deep second-degree burn wounds were induced in rats and the effects of daily HeNe irradiation at 1.2 and 2.4 J/cm(2) were assessed in comparison to 0.2% nitrofurazone cream. The number of macrophages at day 16, and the depth of new epidermis at day 30, was significantly less in the laser treated groups in comparison with control and nitrofurazone treated groups. Additionally, infections with S. epidermidis and S. aureus were significantly reduced [75]. Growth factor secretion by LLL and its apparent regenerative activities have stimulated studies in radiation-induced mucositis. A 30 patient randomized trial of carcinoma patients treated by radiotherapy alone (65 Gy at a rate of 2 Gy/fraction, 5 fractions per week) without prior surgery or concomitant chemotherapy suffering from radiation-induced mucositis was performed using a HeNe 60 mW laser. Grade 3 mucositis occured with a frequency of 35.2% in controls and at 7.6% of treated patients. Furthermore, a decrease in “severe pain” (grade 3) was observed in that 23.8% in the control group experienced this level of pain, as compared to 1.9% in the treatment group. [76]. A subsequent study reported similar effects [77]. Healing ability of lasers was also observed in a study of patients with gingival flap incisions. Fifty-eight extraction patients had one of two gingival flap incisions lased with a 1.4 mw helium-neon (670 nm) at 0.34 J/cm(2). Healing rates were evaluated clinically and photographically. Sixty-nine percent of the irradiated incisions healed faster than the control incisions. No significant difference in healing was noted when patients were compared by age, gender, race, and anatomic location of the incision [78]. Another study evaluating healing effects of LLL in dental practice examined 48 patients subjected to surgical removal of their lower third molars. Treated patients were administered Ga—Al—As diode generated 808 nm at a dose of 12 J. The study demonstrated that extraoral LLLT is more effective than intraoral LLLT, which was more effective than control for the reduction of postoperative trismus and swelling after extraction of the lower third molar [79]. Given the predominance of data supporting fibroblast proliferative ability and animal wound healing effects of LLL therapy, a clinical trial was performed on healing of ulcers. In a double-blinded fashion 23 diabetic leg ulcers from 14 patients were divided into two groups. Phototherapy was applied (<1.0 J cm(-2)) twice per week, using a Dynatron Solaris 705(R) LED device that concurrently emits 660 and 890 nm energies. At days 15, 30, 45, 60, 75, and 90 dmean ulcer granulation and healing rates were significantly higher for the treatment group as compared to control. By day 90, 58.3% of the ulcers in the LLL treated group were fully healed and 75% achieved 90-100% healing. In the placebo group only one ulcer healed fully [58]. As previously mentioned, LLL appears to have some angiogenic activity. One of the major problems in coronary artery disease is lack of collateralization. In a 39 patient study advanced CAD, two sessions of irradiation of low-energy laser light on skin in the chest area from helium-neon B1 lasers. The time of irradiation was 15 minutes while operations were performed 6 days a week for one month. Reduction in Canadian Cardiology Society (CCS) score, increased exercise capacity and time, less frequent angina symptoms during the treadmill test, longer distance of 6-minute walk test and a trend towards less frequent 1 mm ST depression lasting 1 min during Holter recordings was noted after therapy [80]. Perhaps one of the largest clinical trials with LLL was the NEST trial performed by Photothera. In this double blind trial 660 stroke patients were recruited and randomized: 331 received LLL and 327 received sham. No prespecified test achieved significance, but a post hoc analysis of patients with a baseline National Institutes of Health Stroke Scale score of <16 showed a favorable outcome at 90 days on the primary end point (P<0.044) [81]. In one embodiment of the invention, growth factors are added to the cells in culture, prior to, concurrent with, or subsequently the LLL treatment, said growth factors may include FGF-2, EGF, FGF-4, FGF-6, FGF-7, HB-EGF, HGF, IGFBP-1, IGFBP-2, IGFBP-3, IGFPB-4, IGFBP-6, IGF-I, IGF-I SR, IGF-II, M-CSF, M-CSF R, PDGF R.alpha., PDGF-R.beta., PDAF-AA, PDGF-AB, PDGF-BB, PIGF, SCF, TGF-.beta.3, VEGF, or VEGF R2.

In one embodiment of the invention, adipose derived stem cells are administered intravenously at a concentration of 1 million cells per kilogram body weight and the skin surface where rejuvenation is desired is stimulated with LLL using a HeNe radiation of fluency 2.2 J/cm(2) for 3 min twice daily for 14 days. Other means of selectively inducing homing of adipose derived stem cells to the area of need include introduction of a CXCR4 agonist such as stromal derived factor (SDF)-1. Said SDF-1 may be administered as a protein, or as a nucleic acid. In some embodiments electroporation is used to induce topical delivery of SDF-1. In other embodiments, said SDF-1 or a suitable chemoattractant is applied to the area of the skin where regeneration is desired, together with at least one cosmetically or pharmaceutically acceptable ingredient selected from the group consisting of DNA protection agents, DNA repair agents, stem cell protecting agents, agents inhibiting neuronal exocytosis, anticholinergic agents, agents inhibiting muscular contraction, antiaging agents, anti-wrinkle agents, antiperspirant agents, anti-inflammatory and/or analgesic agents, anti-itching agents, calming agents, anesthetic agents, inhibitors of acetylcholine-receptor aggregation, inhibitors of acetylcholinesterase, skin relaxant agents, melanin synthesis stimulating or inhibiting agents, whitening or depigmenting agents, propigmenting agents, self-tanning agents, NO-synthase inhibiting agents, 5.alpha.-reductase inhibiting agents, lysyl- and/or prolyl hydroxylase inhibiting agents, antioxidants, free radical scavengers and/or agents against atmospheric pollution, reactive carbonyl species scavengers, anti-glycation agents, detoxifying agents, antihistamine agents, antiviral agents, antiparasitic agents, emulsifiers, emollients, organic solvents, liquid propellants, skin conditioners, humectants, substances which retain moisture, alpha hydroxy acids, beta hydroxy acids, moisturizers, hydrolytic epidermal enzymes, vitamins, amino acids, proteins, pigments, colorants, dyes, biopolymers, gelling polymers, thickeners, surfactants, softening agents, emulsifiers, binding agents, preservatives, agents able to reduce or treat the bags under the eyes, exfoliating agents, keratolytic agents, desquamating agents, antimicrobial agents, antifungal agents, fungistatic agents, bactericidal agents, bacteriostatic agents, agents stimulating the synthesis of dermal or epidermal macromolecules and/or capable of inhibiting or preventing their degradation, collagen synthesis-stimulation agents, elastin synthesis-stimulation agents, decorin synthesis-stimulation agents, laminin synthesis-stimulation agents, defensin synthesis-stimulating agents, chaperone synthesis-stimulating agents, cAMP synthesis-stimulating agents, AQP-3 modulating agents, aquaporin synthesis-stimulating agents, proteins of the aquaporin family, hyaluronic acid synthesis-stimulating agents, glycosaminoglycan synthesis-stimulating agents, fibronectin synthesis-stimulating agents, sirtuin synthesis-stimulating agents, sirtuin-activating agents, heat shock proteins, heat shock protein synthesis-stimulating agents, agents stimulating the synthesis of lipids and components of the stratum corneum, ceramides, fatty acids, agents that inhibit collagen degradation, agents that inhibit matrix metalloproteinase, agents that inhibit elastin degradation, agents that inhibit serine proteases, agents stimulating fibroblast proliferation, agents stimulating keratinocyte proliferation, agents stimulating adipocyte proliferation, agents stimulating melanocyte proliferation, agents stimulating keratinocyte differentiation, agents stimulating or delaying adipocyte differentiation, antihyperkeratosis agents, comedolytic agents, anti-psoriatic agents, stabilizers, agents for the treatment and/or care of sensitive skin, firming agents, anti-stretch mark agents, binding agents, agents regulating sebum production, lipolytic agents or agents stimulating lipolysis, adipogenic agents, agents modulating PGC-1.alpha. expression, agents modulating the activity of PPAR.gamma., agents which increase or reduce the triglyceride content of adipocytes, anti-cellulite agents, agents which inhibit PAR-2 activity, agents stimulating healing, coadjuvant healing agents, agents stimulating reepithelialization, coadjuvant reepithelialization agents, cytokine growth factors, agents acting on capillary circulation and/or microcirculation, agents stimulating angiogenesis, agents that inhibit vascular permeability, venotonic agents, agents acting on cell metabolism, agents to improve dermal-epidermal junction, agents inducing hair growth, hair growth inhibiting or retardant agents, agents delaying hair loss, preservatives, perfumes, cosmetic and/or absorbent and/or body odor-masking deodorants, chelating agents, plant extracts, essential oils, marine extracts, agents obtained from a biotechnological process, mineral salts, cell extracts, sunscreens and organic or mineral photoprotective agents active against ultraviolet A and/or B rays and/or infrared A rays, and mixtures thereof.

In some embodiments of the invention, mesenchymal stem cells derived from stromal vascular fraction are treated with epigenetic modifying agents to promote a dedifferentiated state prior to administration intradermally. Said agents include but are not limited to, 5-Azacytidine. 5-Aza-20-deoxycytidine, Arabinosyl-5-azacytidine, 5-6-Dihydro-5-azacytidine, 5-Fluoro-20-deoxycytidine, EGX30P, Epigallocatechin-3-gallate, Green tea polyphenol, Hydralazine, MG98, Procainamide, Procaine, and Zebularine. Examples of other histone deacetylase inhibitors include, but are not limited to Apicidin, Butyrates. Phenylbutyrate, m-Carboxycinnamic acid bishydroxamide (CBHA). Cyclic hydroxamic-acid-containing peptide 1 (CHAP 1), TSA-Trapoxin Hybrid, Depudecin Epoxide, Depsipeptide FR901228, Benzamidine, LAQ824, Oxamflatin, MGCD0103, PXD101. Pyroxamide, Suberic Bishydroxamic Acid (SBHA), Suberoylanilide Hydroxamic Acid (SAHA), Trichostatin A (TSA), Trapoxin A, and Valproic acid. Other agents that enhance self-renewal may be utilized such as inhibitors of GSK-3, one such inhibitor being lithium. Formulations and use of lithium for stimulation of stem cells are described in the following papers which are incorporated by reference [82-86]. Without being bound to theory, addition of lithium and salts thereof may be incorporated into the cosmetic mixture with the purpose of preventing apoptosis of progenitor cells [87]. Additionally, combinations of epigenetic acting agents together with lithium are envisioned within the practice of the invention to stimulate effects of conditioned media, or to enhance ability of cells to generate conditioned media. Previous combinations of the epigenetic modulator valproic acid with lithium have been published, which can guide one of skill in the art in practice of the invention [88, 89]. Use of lithium to induce dedifferentiation or rejuvenation of cells has previously been performed in experiments in which lithium can enhance inducible pluripotent stem cell generation [90], the generation of these cells being essentially a dedifferentiation of adult stem cells into a pluripotent state.

When cells of the invention (or extracts thereof) are administered to the skin surface in the form for external use, the skin surface can be heated with a heating means prior to, simultaneously with, and/or after the administration of the skin care product, cosmetic or medicament, to increase the skin temperature to a temperature of 38.degree. C. or higher, but not harmful to the skin. For example, (i) the skin care product, cosmetic or medicament of the present invention may be applied or sprayed onto the skin surface and then the skin surface is heated immediately; (ii) the skin care product, cosmetic or medicament of the present invention may be applied or sprayed onto the skin surface for 15 to 30 minutes, and then the skin surface is heated; (iii) the skin surface is heated for 3 to 5 minutes, and then the skin care product, cosmetic or medicament of the present invention is applied or sprayed onto the skin surface; or (iv) the skin surface is heated to appropriately increase the skin surface temperature and maintain at the increased temperature, while the skin care product, cosmetic or medicament of the present invention is applied or sprayed onto the skin surface during the heating period. The skin surface can be heated with a contact heating means or a non-contact heating means. For example, a suitable contact heating means includes, but is not limited to applying a facial mask to the skin surface, or placing a heat pack, a hot towel, a heating pad, or a heating plate on the skin surface, to appropriately increase the skin temperature through the use of the facial mask, heating pack, hot towel, heating pad, or a heating plate. A suitable non-contact heating means includes the use of a steam engine (such as beauty making ion steamer) and a heating lamp, to heat the skin surface and increase the skin temperature by hot steam released from a steam machine or light irradiated from a heating lamp. In some embodiments of the present invention, a heating pad was used to increase the skin surface temperature by a contact heating means. The heating operation shall heat the skin surface to a temperature harmless to the skin, for example, a temperature of about 38.degree. C. or a higher suitable temperature ranging from such as 38.degree. C. to 50.degree. C. In some embodiments of the present invention, the skin surface temperature was increased to about 39.degree. C. and maintained for about 1 to 2 hours. In the present invention, because the mesenchymal stem cell (or extract thereof) is used and the effective components contained therein are directly utilized to provide the desired effect, the usage amount of the mesenchymal stem cell extract can be controlled more easily, and this is different from the traditional stem cell therapy that provides the desired effect by applying living cells to secrete the effective components in a subject. Depending on the requirements of the subject, the skin care product, cosmetic or medicament manufactured by the mesenchymal stem cell extract of the present invention can be applied with various administration frequencies, such as once a day, several times a day or once for several days, etc. For example, when applying to the skin surface for repairing skin aging, the dosage of the skin care product, cosmetic or medicament may range from about 0.01 ml (as the mesenchymal stem cell extract)/cm.sup.2 to about 1 ml (as the mesenchymal stem cell extract)/cm.sup.2 per day, and preferably from about 0.05 ml (as the mesenchymal stem cell extract)/cm.sup.2 to about 0.5 ml (as the mesenchymal stem cell extract)/cm.sup.2 per day, wherein the unit “ml/cm.sup.2” refers to the dosage required per cm.sup.2-surface area of the treated subject. However, for subjects with more severe skin aging conditions, the dosage can be increased to several times or several tens of times, depending on the practical requirements. In an embodiment of using the mesenchymal stem cell extract of the present invention for repairing skin aging, the dosage of the skin care product, cosmetic or medicament is about 0.1 ml (as the mesenchymal stem cell extract)/cm.sup.2 per day.

In some embodiments of the invention, biologically active agents are added together with said stem cells intradermally, said biologically active agents comprises an active agent selected from the group consisting of a collagen (types I-V), proteoglycans, glycosaminoglycans (GAGs), glycoproteins, cytokines, cell-surface associated proteins, cell adhesion molecules (CAM), endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE). Furthermore, in some aspects of the invention, retention and in vivo activation of said mesenchymal stem cells, or stromal vascular fraction cells is desired. In these situations, said cells are administered together with extracellular matrix (ECM) composition including at least one ECM material selected from the group consisting of small intestine submucosa (SIS), urinary bladder submucosa (UBS), urinary basement membrane (UBM), liver basement membrane (LBM), stomach submucosa (SS), mesothelial tissue, subcutaneous extracellular matrix, large intestine extracellular matrix, placental extracellular matrix, ornamentum extracellular matrix, heart extracellular matrix and lung extracellular matrix; administering inciting event means to a target skin location on the subject to induce at least one inciting event at said target skin location; and administering a therapeutically effective amount of said ECM composition to said target skin location. Furthermore, in some embodiments, anti-inflammatory agents are added together with said stromal vascular fraction cells or mesenchymal stem cells derived thereof. Said anti-inflammatory agents include alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sennetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

### Example 1: Stimulation of Skin Regenerating Factors from Adipose Mesenchymal Stem Cells by Treatment with TLR-2 Agonist Zymosan

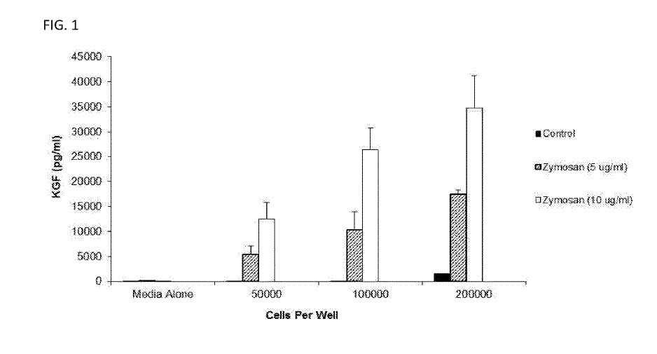

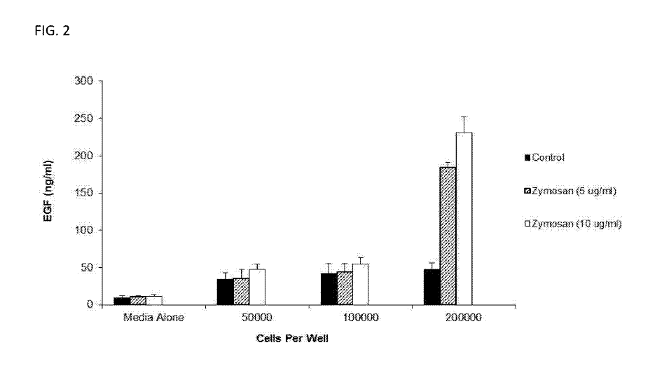

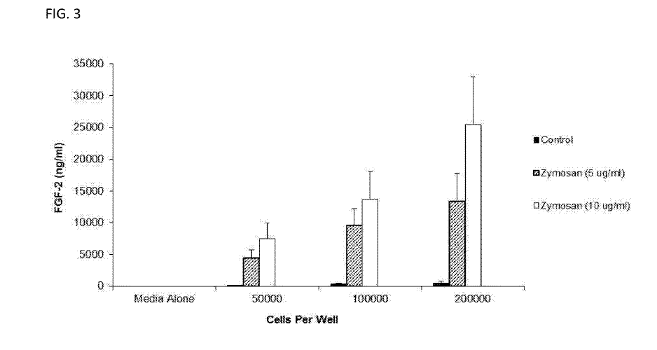

Adipose mesenchymal stem cells are isolated as follows: a) Using aseptic technique and with local anesthesia, the infraumbilical region is infiltrated with 0.5% Xylocaine with 1:200,000 epinephrine; b) After allowing 10 minutes for hemostasis, a 4 mm cannula attached to a 60 cc Toomey syringe is used to aspirate 500 cc of adipose tissue in a circumincisional radiating technique; c) As each of 9 syringes are filled, said syringes are removed from the cannula, capped, and exchanged for a fresh syringe in a sterile manner within the sterile field; d) Using aseptic laboratory technique, the syringe-filled lipoaspirate are placed into two sterile 500 mL centrifuge containers and washed three times with sterile Dulbecco's phosphate-buffered saline to eliminate erythrocytes; e) ClyZyme/PBS (7 mL/500 mL) is added to the washed lipoaspirate using a 1:1 volume ratio; f) The centrifuge containers are sealed and placed in a 37° C. shaking water bath for one hour then centrifuged for 5 min at 300 rcf; g) Following centrifugation, the stromal cells are resuspended within Isolyte in separate sterile 50 mL centrifuge tubes; g) The tubes are centrifuged for 5 min. at 300 rcf and the Isolyte is removed, leaving cell pellet; h) The pellets are resuspended in 40 ml of Isolyte, centrifuged again for 5 min at 300 rcf. The supernatant is again be removed; i) The cell pellets are combined and filtered through 100 μm cell strainers into a sterile 50 ml centrifuge tube and centrifuged for 5 min at 300 rcf and the supernatant removed, leaving the pelleted adipose stromal cells. Cells are subsequently grown in media containing 57% DMEM/F-12, 40% MCDB-201, 2% fetal calf serum, 10 ng/ml epidermal growth factor, 10 ng/ml platelet-derived growth factor BB, 100 U/ml penicillin, and 100 g/ml streptomycin. Once adherent cells were more than 70% confluent, cells are detached with 0.125% trypsin and 0.01% EDTA, and replated at a 1:3 dilution under the same culture conditions. Cells are cultured in 96 well plates and assessed for the cytokines indicted below in response to zymosan at concentrations of 5 and 10 ug/ml. As observed, and increase in skin regenerative factors was observed. FIG. 1 KGF; FIG. 2 EGF; and FIG. 3 FGF-beta.

### REFERENCES

1. Henson, P. M., D. L. Bratton, and V. A. Fadok, The phosphatidylserine receptor: a crucial molecular switch? Nat Rev Mol Cell Biol, 2001. 2(8): p. 627-33.

2. Fadok, V. A., et al., A receptor for phosphatidylserine-specific clearance of apoptotic cells. Nature, 2000. 405(6782): p. 85-90.

3. Fadok, V. A., et al., Exposure of phosphatidylserine on the surface of apoptotic lymphocytes triggers specific recognition and removal by macrophages. J Immunol, 1992. 148(7): p. 2207-16.

4. Shi, D., et al., Artificial phosphatidylserine liposome mimics apoptotic cells in inhibiting maturation and immunostimulatory function of murine myeloid dendritic cells in response to 1-chloro-2,4-dinitrobenze in vitro. Arch Dermatol Res, 2007. 299(7): p. 327-36.

5. Guillaume-Jugnot, P., et al., Autologous adipose-derived stromal vascular fraction in patients with systemic sclerosis: 12-month follow-up. Rheumatology (Oxford), 2016. 55(2): p. 301-6.

6. Trivisonno, A., et al., Harvest of superficial layers of fat with a microcannula and isolation of adipose tissue-derived stromal and vascular cells. Aesthet Surg J, 2014. 34(4): p. 601-13.

7. Tzouvelekis, A., et al., A prospective, non-randomized, no placebo-controlled, phase Ib clinical trial to study the safety of the adipose derived stromal cells-stromal vascular fraction in idiopathic pulmonary fibrosis. J Transl Med, 2013. 11: p. 171.

8. Gentile, P., et al., A comparative translational study: the combined use of enhanced stromal vascular fraction and platelet-rich plasma improves fat grafting maintenance in breast reconstruction. Stem Cells Transl Med, 2012. 1(4): p. 341-51.

9. Lee, S. K., et al., Facial Soft Tissue Augmentation using Autologous Fat Mixed with Stromal Vascular Fraction. Arch Plast Surg, 2012. 39(5): p. 534-9.

10. Aronowitz, J. A. and J. D. Ellenhorn, Adipose stromal vascular fraction isolation: a head-to-head comparison of four commercial cell separation systems. Plast Reconstr Surg, 2013. 132(6): p. 932e-9e.

11. Maiman T. H. Stimulated optical radiation in Ruby. Nature 187:493.

12. Roy, D., Ablative facial resurfacing. Dermatol Clin, 2005. 23(3): p. 549-59,viii.

13. Brown, M. C., An evidence-based approach to patient selection for laser vision correction. J Refract Surg, 2009. 25(7 Suppl): p. S661-7.

14. Brancaleon, L. and H. Moseley, Laser and non-laser light sources for photodynamic therapy. Lasers Med Sci, 2002. 17(3): p. 173-86.

15. Mester, E. S., B., and Tota, J. G. (1967). “Effect of laser on hair growth of mice”. Kiserl Orvostud 19: 628-631.

16. Mester, E., et al., The effect of laser irradiation on the regeneration of muscle fibers (preliminary report). Z Exp Chir, 1975. 8(4): p. 258-62.

17. Posten, W., et al., Low-level laser therapy for wound healing: mechanism and efficacy. Dermatol Surg, 2005. 31(3): p. 334-40.

18. Vladimirov, Y. A., A. N. Osipov, and G. I. Klebanov, Photobiological principles of therapeutic applications of laser radiation. Biochemistry (Mosc), 2004. 69(1): p. 81-90.

19. Hu, W. P., et al., Helium-neon laser irradiation stimulates cell proliferation through photostimulatory effects in mitochondria. J Invest Dermatol, 2007. 127(8): p. 2048-57.

20. Moriyama, Y., et al., In vivo effects of low level laser therapy on inducible nitric oxide synthase. Lasers Surg Med, 2009. 41(3): p. 227-31.

21. Samoilova, K. A., et al., Role of nitric oxide in the visible light-induced rapid increase of human skin microcirculation at the local and systemic levels: II. healthy volunteers. Photomed Laser Surg, 2008. 26(5): p. 443-9.

22. Yamaura, M., et al., Low level light effects on inflammatory cytokine production by rheumatoid arthritis synoviocytes. Lasers Surg Med, 2009. 41(4): p. 282-90.

23. Shiba, H., et al., Neodymium-doped yttrium-aluminium-garnet laser irradiation abolishes the increase in interleukin-6 levels caused by peptidoglycan through the p38 mitogen-activated protein kinase pathway in human pulp cells. J Endod, 2009. 35(3): p. 373-6.

24. Mafra de Lima, F., et al., Low level laser therapy (LLLT): attenuation of cholinergic hyperreactivity, beta(2)-adrenergic hyporesponsiveness and TNF-alpha mRNA expression in rat bronchi segments in E. coli lipopolysaccharide-induced airway inflammation by a NF-kappaB dependent mechanism. Lasers Surg Med, 2009. 41(1): p. 68-74.

25. Aimbire, F., et al., Low level laser therapy (LLLT) decreases pulmonary microvascular leakage, neutrophil influx and IL-1 beta levels in airway and lung from rat subjected to LPS-induced inflammation. Inflammation, 2008. 31(3): p. 189-97.

26. Safavi, S. M., et al., Effects of low-level He—Ne laser irradiation on the gene expression of IL-1beta, TNF-alpha, IFN-gamma, TGF-beta, bFGF, and PDGF in rat's gingiva. Lasers Med Sci, 2008. 23(3): p. 331-5.

27. Saygun, I., et al., Effects of laser irradiation on the release of basic fibroblast growth factor (bFGF), insulin like growth factor-1 (IGF-1), and receptor of IGF-1 (IGFBP3) from gingival fibroblasts. Lasers Med Sci, 2008. 23(2): p. 211-5.

28. Schwartz, F., et al., Effect of helium/neon laser irradiation on nerve growth factor synthesis and secretion in skeletal muscle cultures. J Photochem Photobiol B, 2002. 66(3): p. 195-200.

29. Yu, W., J. O. Naim, and R. J. Lanzafame, The effect of laser irradiation on the release of bFGF from 3T3 fibroblasts. Photochem Photobiol, 1994. 59(2): p. 167-70.

30. Zungu, I. L., D. Hawkins Evans, and H. Abrahamse, Mitochondrial responses of normal and injured human skin fibroblasts following low level laser irradiation—an in vitro study. Photochem Photobiol, 2009. 85(4): p. 987-96.

31. Wu, S., et al., High fluence low-power laser irradiation induces mitochondrial permeability transition mediated by reactive oxygen species. J Cell Physiol, 2009. 218(3): p. 603-11.

32. Lan, C. C., et al., Low-energy helium-neon laser induces melanocyte proliferation via interaction with type IV collagen: visible light as a therapeutic option for vitiligo. Br J Dermatol, 2009. 161(2): p. 273-80.

33. Karu, T., Photobiology of low-power laser effects. Health Phys, 1989. 56(5): p. 691-704.

34. Tiphlova, O. and T. Karu, Role of primary photoacceptors in low-power laser effects: action of He—Ne laser radiation on bacteriophage T4-Escherichia coli interaction. Lasers Surg Med, 1989. 9(1): p. 67-9.

35. Karu, T. I., Mitochondrial signaling in mammalian cells activated by red and near-IR radiation. Photochem Photobiol, 2008. 84(5): p. 1091-9.

36. Zhang, L., et al., Low-power laser irradiation inhibiting Abeta25-35-induced PC12 cell apoptosis via PKC activation. Cell Physiol Biochem, 2008. 22(1-4): p. 215-22.

37. Aimbire, F., et al., Low-level laser therapy decreases levels of lung neutrophils anti-apoptotic factors by a NF-kappaB dependent mechanism. Int Immunopharmacol, 2008. 8(4): p. 603-5.

38. Lipovsky, A., Y. Nitzan, and R. Lubart, A possible mechanism for visible light-induced wound healing. Lasers Surg Med, 2008. 40(7): p. 509-14.

39. Ignatieva, N., et al., Effects of laser irradiation on collagen organization in chemically induced degenerative annulus fibrosus of lumbar intervertebral disc. Lasers Surg Med, 2008. 40(6): p. 422-32.

40. Silveira, L.B., et al., Investigation of mast cells in human gingiva following low-intensity laser irradiation. Photomed Laser Surg, 2008. 26(4): p. 315-21.

41. Coombe, A. R., et al., The effects of low level laser irradiation on osteoblastic cells. Clin Orthod Res, 2001. 4(1): p. 3-14.

42. Gregoraszczuk, E., J. W. Dobrowolski, and J. Galas, Effect of low intensity laser beam on steroid dehydrogenase activity and steroid hormone production in cultured porcine granulosa cells. Folia Histochem Cytochem (Krakow), 1983. 21(2): p. 87-92.

43. Yu, H. S., et al., Low-energy helium-neon laser irradiation stimulates interleukin-1 alpha and interleukin-8 release from cultured human keratinocytes. J Invest Dermatol, 1996. 107(4): p. 593-6.

44. Mvula, B., et al., The effect of low level laser irradiation on adult human adipose derived stem cells. Lasers Med Sci, 2008. 23(3): p. 277-82.

45. Mvula, B., T. J. Moore, and H. Abrahamse, Effect of low-level laser irradiation and epidermal growth factor on adult human adipose-derived stem cells. Lasers Med Sci. 25(1): p. 33-9.

46. Hawkins, D. H. and H. Abrahamse, The role of laser fluence in cell viability, proliferation, and membrane integrity of wounded human skin fibroblasts following helium-neon laser irradiation. Lasers Surg Med, 2006. 38(1): p. 74-83.

47. Houreld, N. and H. Abrahamse, In vitro exposure of wounded diabetic fibroblast cells to a helium-neon laser at 5 and 16 J/cm2. Photomed Laser Surg, 2007. 25(2): p. 78-84.

48. Smol'yaninova, N. K., et al., Effects of He—Ne laser irradiation on chromatin properties and synthesis of nucleic acids in human peripheral blood lymphocytes. Biomed Sci, 1991. 2(2): p. 121-6.

49. Funk, J. O., et al., Helium-neon laser irradiation induces effects on cytokine production at the protein and the mRNA level. Exp Dermatol, 1993. 2(2): p. 75-83.

50. Funk, J. O., A. Kruse, and H. Kirchner, Cytokine production after helium-neon laser irradiation in cultures of human peripheral blood mononuclear cells. J Photochem Photobiol B, 1992. 16(3-4): p. 347-55.

51. Gulsoy, M., et al., The biological effects of 632.8-nm low energy He—Ne laser on peripheral blood mononuclear cells in vitro. J Photochem Photobiol B, 2006. 82(3): p. 199-202.

52. Novoselova, E. G., et al., [Effect of low-intensity laser radiation (632.8 nm) on immune cells isolated from mice] . Biofizika, 2006. 51(3): p. 509-18.

53. Dube, A., H. Bansal, and P. K. Gupta, Modulation of macrophage structure and function by low level He—Ne laser irradiation. Photochem Photobiol Sci, 2003. 2(8): p. 851-5.

54. Hemvani, N., D. S. Chitnis, and N. S. Bhagwanani, Helium-neon and nitrogen laser irradiation accelerates the phagocytic activity of human monocytes. Photomed Laser Surg, 2005. 23(6): p. 571-4.

55. Moustsen, P. A., et al., [Laser treatment of sinusitis in general practice assessed by a double-blind controlled study]. Ugeskr Laeger, 1991. 153(32): p. 2232-4.

56. Shen, X., et al., Effect of combined laser acupuncture on knee osteoarthritis: a pilot study. Lasers Med Sci, 2009. 24(2): p. 129-36.

57. Ekim, A., et al., Effect of low level laser therapy in rheumatoid arthritis patients with carpal tunnel syndrome. Swiss Med Wkly, 2007. 137(23-24): p. 347-52.

58. Minatel, D. G., et al., Phototherapy promotes healing of chronic diabetic leg ulcers that failed to respond to other therapies. Lasers Surg Med, 2009. 41(6): p. 433-41.

59. Bocci, V., V. Travagli, and I. Zanardi, May oxygen-ozone therapy improves cardiovascular disorders? Cardiovasc Hematol Disord Drug Targets, 2009. 9(2): p. 78-85.

60. Bocci, V., et al., The ozone paradox: ozone is a strong oxidant as well as a medical drug. Med Res Rev, 2009. 29(4): p. 646-82.

61. Re, L., et al., Ozone therapy: clinical and basic evidence of its therapeutic potential. Arch Med Res, 2008. 39(1): p. 17-26.

62. Damera, G., et al., Ozone modulates IL-6 secretion in human airway epithelial and smooth muscle cells. Am J Physiol Lung Cell Mol Physiol, 2009. 296(4): p. L674-83.

63. Manzer, R., et al., Ozone exposure of macrophages induces an alveolar epithelial chemokine response through IL-1alpha. Am J Respir Cell Mol Biol, 2008. 38(3): p. 318-23.

64. McDonald, R. J. and J. Usachencko, Neutrophils injure bronchial epithelium after ozone exposure. Inflammation, 1999. 23(1): p. 63-73.

65. Rodriguez, Z. Z., et al., Preconditioning with ozone/oxygen mixture induces reversion of some indicators of oxidative stress and prevents organic damage in rats with fecal peritonitis. Inflamm Res, 2009.

66. Zamora, Z. B., et al., Effects of ozone oxidative preconditioning on TNF-alpha release and antioxidant-prooxidant intracellular balance in mice during endotoxic shock. Mediators Inflamm, 2005. 2005(1): p. 16-22.

67. Borrego, A., et al., Protection by ozone preconditioning is mediated by the antioxidant system in cisplatin-induced nephrotoxicity in rats. Mediators Inflamm, 2004. 13(1): p. 13-9.

68. Martinez-Sanchez, G., et al., Therapeutic efficacy of ozone in patients with diabetic foot. Eur J Pharmacol, 2005. 523(1-3): p. 151-61.

69. Surinchak, J. S., et al., Effects of low-level energy lasers on the healing of full-thickness skin defects. Lasers Surg Med, 1983. 2(3): p. 267-74.

70. Broadley, C., et al., Low-energy helium-neon laser irradiation and the tensile strength of incisional wounds in the rat. Wound Repair Regen, 1995. 3(4): p. 512-7.

71. Ghamsari, S. M., et al., Histopathological effect of low-level laser therapy on sutured wounds of the teat in dairy cattle. Vet Q, 1996. 18(1): p. 17-21.

72. Ghamsari, S. M., et al., Evaluation of low level laser therapy on primary healing of experimentally induced full thickness teat wounds in dairy cattle. Vet Surg, 1997. 26(2): p. 114-20.

73. Pinfildi, C. E., et al., Helium-neon laser in viability of random skin flap in rats. Lasers Surg Med, 2005. 37(1): p. 74-7.

74. Maiya, G. A., P. Kumar, and L. Rao, Effect of low intensity helium-neon (He—Ne) laser irradiation on diabetic wound healing dynamics. Photomed Laser Surg, 2005. 23(2): p. 187-90.

75. Bayat, M., et al., Effect of low-level laser therapy on the healing of second-degree burns in rats: a histological and microbiological study. J Photochem Photobiol B, 2005. 78(2): p. 171-7.

76. Bensadoun, R. J., et al., Low-energy He/Ne laser in the prevention of radiation-induced mucositis. A multicenter phase III randomized study in patients with head and neck cancer. Support Care Cancer, 1999. 7(4): p. 244-52.

77. Arun Maiya, G., M. S. Sagar, and D. Fernandes, Effect of low level helium-neon (He—Ne) laser therapy in the prevention & treatment of radiation induced mucositis in head & neck cancer patients. Indian J Med Res, 2006. 124(4): p. 399-402.

78. Neiburger, E. J., Rapid healing of gingival incisions by the helium-neon diode laser. J Mass Dent Soc, 1999. 48(1): p. 8-13, 40.

79. Aras, M. H. and M. Gungormus, Placebo-controlled randomized clinical trial of the effect two different low-level laser therapies (LLLT)-intraoral and extraoral-on trismus and facial swelling following surgical extraction of the lower third molar. Lasers Med Sci, 2009.

80. Zycinski, P., et al., Laser biostimulation in end-stage multivessel coronary artery disease—a preliminary observational study. Kardiol Pol, 2007. 65(1): p. 13-21; discussion 22-3.

81. Zivin, J. A., et al., Effectiveness and safety of transcranial laser therapy for acute ischemic stroke. Stroke, 2009. 40(4): p. 1359-64.

82. Dong, B. T., et al., Lithium enhanced cell proliferation and differentiation of mesenchymal stem cells to neural cells in rat spinal cord. Int J Clin Exp Pathol, 2015. 8(3): p. 2473-83.

83. Bernick, J., et al., Parameters for lithium treatment are critical in its enhancement of fracture-healing in rodents. J Bone Joint Surg Am, 2014. 96(23): p. 1990-8.

84. Zhu, Z., et al., Lithium stimulates human bone marrow derived mesenchymal stem cell proliferation through GSK-3beta-dependent beta-catenin/Wnt pathway activation. FEBS J, 2014. 281(23): p. 5371-89.

85. Tsai, H. L., et al., Wnts enhance neurotrophin-induced neuronal differentiation in adult bone-marrow-derived mesenchymal stem cells via canonical and noncanonical signaling pathways. PLoS One, 2014. 9(8): p. e104937.

86. Yoneyama, M., et al., Lithium promotes neuronal repair and ameliorates depression-like behavior following trimethyltin-induced neuronal loss in the dentate gyrus. PLoS One, 2014. 9(2): p. e87953.

87. Cabrera, O., et al., Lithium protects against glucocorticoid induced neural progenitor cell apoptosis in the developing cerebellum. Brain Res, 2014. 1545: p. 54-63.

88. Walasek, M. A., et al., The combination of valproic acid and lithium delays hematopoietic stem/progenitor cell differentiation. Blood, 2012. 119(13): p. 3050-9.

89. Tsai, L. K., et al., Mesenchymal stem cells primed with valproate and lithium robustly migrate to infarcted regions and facilitate recovery in a stroke model. Stroke, 2011. 42(10): p. 2932-9.

90. Wang, Q., et al., Lithium, an anti-psychotic drug, greatly enhances the generation of induced pluripotent stem cells. Cell Res, 2011. 21(10): p. 1424-35.

1. A method of treating skin so that the appearance of the skin is improved, said method comprised of: a) extracting adipose cells from said patient in need of skin improvement; b) obtaining the stromal vascular fraction of said adipose tissue; c) exposing said stromal vascular fraction of said adipose tissue to one or more conditions capable of enhancing regenerative activity of cells in said stromal vascular fraction of said adipose tissue; d) administering said cells back to the said patient.

2. The method of claim 1, wherein said skin in need of improvement is selected from a group comprising of: a) aged skin; b) skin with wrinkles; c) sun damaged skin; d) scarred skin; e) hypopigmented skin; and f) skin damaged by skin disorders.

3. The method of claim 1, wherein stromal vascular fraction cells is performed by the following steps: a) Using aseptic technique and with local anesthesia, the infraumbilical region is infiltrated with 0.5% Xylocaine with 1:200,000 epinephrine; b) After allowing 10 minutes for hemostasis, a 4 mm cannula attached to a 60 cc Toomey syringe is used to aspirate 500 cc of adipose tissue in a circumincisional radiating technique; c) As each of 9 syringes are filled, said syringes are removed from the cannula, capped, and exchanged for a fresh syringe in a sterile manner within the sterile field; d) Using aseptic laboratory technique, the syringe-filled lipoaspirate are placed into two sterile 500 mL centrifuge containers and washed three times with sterile Dulbecco's phosphate-buffered saline to eliminate erythrocytes; e) ClyZyme/PBS (7 mL/500 mL) is added to the washed lipoaspirate using a 1:1 volume ratio; f) The centrifuge containers are sealed and placed in a 37° C. shaking water bath for one hour then centrifuged for 5 min at 300 rcf; g) Following centrifugation, the stromal cells are resuspended within Isolyte in separate sterile 50 mL centrifuge tubes; g) The tubes are centrifuged for 5 min. at 300 rcf and the Isolyte is removed, leaving cell pellet; h) The pellets are resuspended in 40 ml of Isolyte, centrifuged again for 5 min at 300 rcf. The supernatant is again be removed; i) The cell pellets are combined and filtered through 100 m cell strainers into a sterile 50 ml centrifuge tube and centrifuged for 5 min at 300 rcf and the supernatant removed, leaving the pelleted adipose stromal cells.

4. The method of claim 1, wherein said adipose derived cells are positively selected for a marker chosen from a group comprising of: a) CD105; b) CD73; c) CD44; d) CD90; e) VEGFR2; and f) TEM-1 and lacking expression of markers chosen from a group comprising of: a) HLA-DR; b) CD45; and c) CD14.

5. The method of claim 4, wherein said activity of said administered cells is selected from a group comprising of: a) enhanced cytokine production; b) enhanced ability to differentiate into cells of the pulmonary architecture; c) augmented ability to produce antiapoptic factors; d) increased angiogenic activity; e) inhibition of inflammatory cytokine production; and f) inhibition of fibrotic activity.

6. The method of claim 5 wherein said laser irradiation is administered by a light source between approximately 100 .mu.W/cm.sup.2 to approximately 10 W/cm.sup.2.

7. The method of claim 1, wherein said cells are administered intradermally.

8. The method of claim 1, wherein said cells are administered systemically.

9. The method of claim 8, wherein said patient receiving cells administered systemically is further treated with an agent or plurality of agents capable of achieving stem cell retention to the dermal area where therapeutic effects are desired.

10. The method of claim 9, wherein said agents capable of achieving stem cell retention are selected from a group comprising of: a) stromal derived factor-1 (SDF-1); b) vascular endothelial growth factor (VEGF); c) epidermal growth factor (EGF); d) platelet rich plasma; e) brain derived neurotrophic factor (BDNF), f) platelet derived growth factor (PDGF); and g) low level laser irradiation.

11. A method of treating skin so that the appearance of the skin is improved, said method comprised of: a) obtaining cells with regenerative properties; b) exposing said cells to one or more conditions capable of enhancing regenerative activity of said cells cells; d) administering said cells to said patient in need of treatment.

12. The method of claim 11, wherein said cells with regenerative properties are autologous, allogeneic, or xenogeneic to the recipient.

13. The method of claim 11, wherein said cells with regenerative properties are obtained from tissues selected from a group of tissues comprising of: a) adipose tissue; b) bone marrow; c) muscle; d) mobilized peripheral blood; e) hair follicle; f) teeth; and g) periventricular fluid.

14. The method of claim 13, wherein cells possessing regenerative potential are mesenchymal.

15. The method of claim 14, wherein said cells possess ability to produce cytokines selected from a group comprising of: a) FGF-alpha; b) FGF-beta; c) FGF-V; d) EGF; e) IGF; f) VEGF; g) SDF-1; h) PDGF-1; and i) BDNF.

16. The method of claim 15 wherein the step of processing said cells from said adipose tissue so as to concentrate said stem cell component is performed through treatment with an enzyme capable of enriching for stromal vascular fraction cells.

17. The method of claim 13, wherein the step of processing said cells from said bone marrow tissue to concentrate said stem cell component is performed through removal of erythrocytes and granulocytes by use of a density gradient.

18. The method of claim 13, wherein the step of processing said cells from said muscle tissue so as to concentrate said stem cell component is performed through treatment with an enzyme capable of enriching for cells expressing a marker selected from a group of markers comprising of CD13, CD34, CD56 and CD117.

19. The method of claim 13, wherein the step of processing said cells from said mobilized blood so as to concentrate said stem cell component is performed through leukopheresis of a patient who has been mobilized by a mobilizing agent selected from a group comprising of: G-CSF, Mozobil, VEGF, or parathyroid hormone.
